(19) United States
(12) Patent Application Publication
(10) Pub. No.: US 2012/0215184 A1
(43) Pub. Date: Aug. 23, 2012

(54) CYLINDRICAL OCULAR INSERTS

(75) Inventor: Johnson LIM, Edison, NJ (US)

(73) Assignee: Valeant International (Barbados) SRL

(21) Appl. No.: 13/397,185

(22) Filed: Feb. 15, 2012

Related U.S. Application Data
(60) Provisional application No. 61/444,213, filed on Feb. 18, 2011.

(51) Int. Cl. A61F 9/00; B29D 11/00; A61M 35/00

(52) U.S. Cl. 604/290; 604/294; 264/2.7

(57) ABSTRACT

Ocular inserts including a cylindrical body, a lumen and a hydrophilic polymer and strips thereof are suitable for contact with an eye of a subject suffering from an eye disorder. While in contact with an eye, the ocular inserts may be used for releasing various agents, including pharmaceutically active agents, and effecting a treatment of one or more eye disorders.

### CROSS REFERENCE TO RELATED APPLICATIONS

This application claims the benefit of U.S. Provisional Application No. 61/444,213, filed on Feb. 18, 2011, the entire disclosure of which is hereby incorporated by reference for all purposes in its entirety as if fully set forth herein.

### BACKGROUND

Ocular inserts are used to treat a variety of eye disorders. For example, commercially available Lacrisert®, is used to treat dry eye. Lacrisert® is a sterile, translucent, solid rod-shaped, water soluble, ophthalmic insert made of hydroxypropyl cellulose for administration into the inferior cul-de-sac of the eye by a patient or a medical practitioner. Once inserted, the hydroxypropyl cellulose slowly dissolves in the eye over a period of several hours to a day. In the case of dry eye treatment, hydroxypropyl cellulose aids in tear retention by increasing tear viscosity to relieve the symptoms associated with dry eye.

Dry eye may be treated with ocular inserts, eye drops, or punctual plugs, whereas the majority of eye disorders are typically treated with eye drops. Eye drops are used to deliver drug to treat acute and chronic diseases states. Eye drops are used for treating ophthalmic conditions, such as local infections, allergies, glaucoma, pain and inflammation. Approximately 90-95% of topical drops are cleared very quickly from the tear film by drainage into the lacrimal ducts or simply lost from the ocular surface due to poor retention. Some approaches attempt to increase this retention by utilization of either viscous gels or ointments, which may cause blurred vision as a side effect. Sustaining a significant concentration of drug on the surface of the eye and within the cornea and conjunctiva presents a daunting challenge. With current topical treatment modalitie's the delivery of an, consitent quantity of drug, to treat ophthalmic diseases, remains very poor.

While certain eye disorders are treated with ocular inserts, gels or ointments, when placed in the eye, these treatments may lead to blurred vision either via application, or in the case of ocular inserts, they may turn into soft thick lumps or masses and then blur vision. There is a need for improved ocular inserts for releasing pharmaceutically active agents and treat eye disorders.

### SUMMARY

Provided herein are ocular inserts having various shapes. Some are hollow cylinders and others are shaped into strips. The hollow cylinders may vary in length and diameter. The thickness of its walls may also vary, and the shapes of the lumens may also vary, as described in greater detail, below. The strips can be flat or twisted. Methods are also provided for preparing the ocular inserts, as well as methods of using them to release various pharmaceutically active agents and/or treat a variety of eye disorders.

In one aspect, an ocular insert is provided including a cylindrical body having an outer surface; at least an inner surface defining at least a lumen; a proximal end; a distal end; a longitudinal axis defining a length of the cylindrical body from the proximal end to the distal end; a transverse axis, orthogonal to the longitudinal axis and defining a width of the cylindrical body; and a hydrophilic polymer. In one embodiment, the hydrophilic polymer exhibits biodegradable, bioabsorbable, or bioerodable properties. In another embodiment, the ocular insert further includes a pharmaceutically active agent, a dye, a lubricant, or an emollient, and optionally a pharmaceutically acceptable excipient, such as a gelling agent. In another embodiment, a composition is provided including an admixture of the ocular insert provided herein with a pharmaceutically active agent, a dye, a lubricant, an emollient, or other excipient, including a gelling agent.

In another aspect, a method is provided for preparing the ocular insert, the method including extruding a strand of a biodegradable polymer along a longitudinal axis, the strand including at least one continuous lumen extending along the axis; cutting the strand into segments of a length suitable as an ocular insert, wherein the segments each include a distal end and a proximal end.

In another aspect, a method is provided for modulating the dissolution rate of an ocular insert including a cylindrical body having a lumen and a hydrophilic polymer, the method including varying a diameter of the lumen.

In another aspect, a method of treating an eye disorder is provided, the method including depositing an ocular insert provided herein into the conjunctival cul-de-sac of an eye of a subject suffering from one or more eye disorders, wherein the ocular insert has a dissolution rate that is increased in comparison to an ocular insert including the hydrophilic polymer that does not include a lumen.

In another aspect, a method of manufacture is provided which includes contacting an ocular insert (either a hollow cylinder or a strip) with a solvent or a solution to provide a solvated or filled ocular insert. In one embodiment, the method further includes allowing a solvated or filled hollow cylindrical ocular insert to collapse to provide a strip including a biodegradable polymer and, optionally, a payload of a pharmaceutically or pharmacologically active substance.

As used herein, “allowing a solvated or filled hollow cylindrical ocular insert to collapse” refers to simply letting a hollow cylinder remain in contact with a solvent or solution until the hollow cylinder loses its ability to maintain its cylindrical shape. Typically the solvated or filled hollow cylindrical ocular insert will collapse into a either flat or twisted strip of hydrophilic polymer depending on the nature of the inner surface of the starting hollow cylinder or “tube.” In particular, it has been discovered that a tube having a smooth inner surface collapses into a twisted strip, and a tube having a ribbed inner surface collapses into a flat strip. In certain embodiments, a ribbed inner lumen tube is preferred over a smooth tube when a substantially flat strip is desired. In addition, it has been discovered that hollow cylinders or tubes having a length of about 30.5 cm (or about 12 inches) or less sustain their structural integrity in the presence of solvent or filling such that they do not collapse into a twisted or flat strip but, rather, maintain their tubular shapes. A flattened strip or twisted strip exhibits a length and a width, while having a thickness much smaller than the dimensions of its width or length. In another embodiment, an ocular insert can be made to harbor a payload, which may include any number of substances, typically one or more pharmaceutically active agents.

In another aspect, a method of treating an eye disorder includes contacting a strip including a hydrophilic polymer and an optional payload with an eye of a subject in need of such treatment.

In another aspect, a method is provided for releasing a pharmaceutically active agent into a liquid medium, which method includes contacting an ocular insert including a pharmaceutically active agent with a liquid medium, thereby releasing the pharmaceutically active agent into the liquid medium.

In some embodiments, the ocular inserts are capable of releasing a pharmaceutically active agent for extended periods of time ranging from about 1 hour up to about 24 hours or more.

A release of a pharmaceutically active agent from an ocular insert into a liquid medium can be carried out in vitro, in vivo, or ex vivo. When performed in vitro such methods may be used, for example, for identifying active pharmaceuticals agents that can be, or need to be, delivered to an eye of a subject at a certain amount or rate. When performed in vivo or ex vivo, such methods may be used in conjunction with animal models of human eye disorders. When released in vivo in humans and animals, the method may lead to an alleviation of one or more negative effects associated with a pathological condition of the human or animal, especially an ophthalmic condition, for example, involving one or more eye disorders.

### BRIEF DESCRIPTION OF THE DRAWINGS

FIG. 1A-1D are photographs of various views of single lumen containing ocular inserts with a ribbed inner surface.

FIG. 2A-2C illustrate the outer surface, the skived end, and the cross section of a cylindrical ocular insert prepared using a short land die.

FIG. 3A-3C illustrate the outer surface, the skived end, and the cross section of a cylindrical ocular insert, prepared using a long land die.

FIG. 4A illustrates the dissolution (or release) profiles of different batches of ocular insert from smooth tubes, which had been loaded multiple times with a solution of Timoptic (timolol maleate) 0.5% ophthalmic solution. The batches (I-V) differed in their initial amounts of loaded timolol maleate. FIG. 4B (“smooth tube”) and 4C (“ribbed tube”) illustrate the dissolution (or release) profiles of different batches of ocular insert, which had been loaded with either gelling agent-containing formulations (batches 1, 1a and 3-6) or an aqueous solution (batch X2) of Timoptic (timolol maleate). The batches (I-V) differed in their initial amounts of loaded timolol maleate. Three types of gelling agents are exemplified: gellan gum, polyethylene glycol 400 and carboxymethyl cellulose.

FIG. 5A-5B illustrate an ocular insert having a cog-shaped ribbed lumen, and having an outer diameter of 0.051″ (1.3 mm), a first inner diameter of 0.041″ (1.04 mm) and a second inner diameter of 0.021″ (0.53 mm). The numbers on the figure represent the distances from the center of the tube to the line indicated. The wall cross sectional area is 0.0014 in² (0.90 mm²).

FIG. 6A-6B illustrate an ocular insert having a cog-shaped ribbed lumen, and having an outer diameter of 0.051″ (1.3 mm), a first inner diameter of 0.037″ (0.94 mm) and a second inner diameter of 0.025″ (0.64 mm). The numbers on the figure represent the distances from the center of the tube to the line indicated. The wall cross sectional area is 0.0013 in² (0.84 mm²).

### DETAILED DESCRIPTION

In one aspect, an ocular insert is provided including a cylindrical body having an outer surface; at least an inner surface defining at least a lumen; a proximal end; a distal end; a longitudinal axis defining a length of the cylindrical body from the proximal end to the distal end; a transverse axis, orthogonal to the longitudinal axis and defining a width of the cylindrical body; and a hydrophilic polymer. Such ocular inserts may be used in a wide variety of applications including treatment of dry eye and the delivery of active agents to the eye.

The hydrophilic polymer may be biodegradable or bioabsorbable. The hydrophilic polymer may include, but is not limited to, hydroxypropyl cellulose, hydroxypropylmethylcellulose, 2-hydroxyethyl starch, poly(bisphenol A-iminocarbonate), poly(butylenes succinate), poly(butylenes succinate-co-adipate), poly(butylene adipate-co-terephthalate), poly[1,6-bis(p-carboxyphenoxy)hexane], poly[(1,6-bis(p-carboxyphenoxy)hexane)-co-sebacic acid], poly(bis(4-carboxyphenoxy)phosphazene), poly(bis(1,4-dioxapentyl)phosphazene), poly(bis(1-(ethoxycarbonyl)methylamino)phosphazene), poly[bis(1-(ethoxycarbonyl)-2-phenylethylamino)phosphazene], poly[1,4-bis(hydroxyethyl)terephthalate-altethyloxyphosphate], poly[1,4-bis(hydroxyethyl)terephthalate-alt-ethyloxyphosphate]-co-1,4-bis(hydroxyethyl)terephthalate-co-terephthalate, poly(caprolactone) (PCL), polycaprolactone diol, polycaprolactone triol, polycaprolactone-polyglycolide, poly(ε-caprolactone)-block-poly(ethylene glycol)-block-poly(ε-caprolactone) (PCL-b-PEG-b-PCL), polycaprolactone-block-polytetrahydrofuran-block-polycaprolactone, poly(detosu-1.6 HD-t-CDM ortho ester), poly(p-dioxanone) (PDS), polydioxanone-polyglycolide-poly(trimethylene carbonate), Biosyn, poly(DTH imino carbonate), poly(epsilon-caprolactone) (PCL), polyesteramide (PEA), poly(ethylene glycol)-block-poly(ε-caprolactone)methyl ether (PCL-b-PEG), poly(ethylene oxide)-block-polycaprolactone (4-arm), poly(ethylene oxide)-block-polylactide (4-arm), polylactide-block-poly(ethylene glycol)-block-polylactide poly(-ethyl-1-glutamate), polyglycolide (PGA), poly(glycolide-co-caprolactone) Monocryl, poly(glycolide-co-L(−)lactide)Vicryl, polyglycolide-poly(trimethylene carbonate) Maxon, polyglycolide-polycaprolactone-poly(trimethylene carbonate)-polyglycolide Caprosyn, polyhydroxybutyrate (PHB), poly[(R)-3-hydroxybutyric acid], poly(3-hydroxybutyrate-co-3-hydroxyhexanoate) PHBHx, poly(hydroxybutyrate-co-hydroxyoctanoate) PHBOd, poly(3-hydroxybutyrate-co-3-hydroxyoctadecanoate) PHBO, poly(3-hydroxybutyrate-co-3-hydroxyvalerate) PHBV, Biopol, poly[L(−)lactide] (PLA), polylactic acid-polyethylene glycol (PLEG), poly(DL-lactide-co-caprolactone), poly(lactide-co-glycolide) PLGA, poly(L-lactide-co-caprolactone-co-glycolide), (PLGC), poly[(lactide-co-ethylene glycol)-co-ethyloxyphosphate], poly(sebacic acid) (SA), poly(sebacic acid-hexadecandioic acid anhydride), poly(trimethylenecarbonate) (PTMC), poly(hydroxy ester ether), polyhydroxyalkanoates (PHA), poly(L-lactide-co-dl-lactide), L-PLA-DL-PLA (Inion), poly(L-lactide-co-DL-lactide-co-trimethylenecarbonate), L-PLA-D-PLA-TMC (Inion), a cellulosic material, a poly(ester amide), a polyethylene glycol (PEG), a polyhydroxyalkanoate (PHA), polyesters, poly(ortho esters), poly(anhydrides), poly(tyrosine carbonates), poly(imino carbonates), poly(phosphoesters), polyphosphazenes, poly(amino acids), polysaccharides, polyethers, polyamides, polyurethanes, polyalkylenes, polyalkylene oxides, a polyhydroxyalkanoate-polyethylene glycol copolymer. alkylene vinyl acetate copolymer, alkylene vinyl alcohol copolymers, poly(D,L-lactide), poly(L-lactide), polyglycolide, poly(D,L-lactide-co-glycolide), polycaprolactone, poly(D,L-lactide-co-caprolactone), poly(glycolide-co-caprolactone), poly(dioxanone), collagen, chitosan, alginate, polyethylene oxide, polypropylene oxide, polyethylene glycol (PEG), polyvinylpyrrolidone (PVP), poly(n-butyl methacrylate) (PBMA), poly(vinylidene fluoride-co-hexafluoropropene), hydroxymethylcellulose, hydroxyethylcellulose, or a mixture of any two or more thereof.

In one embodiment, the hydrophilic polymer is a cellulosic material, a poly(ester amide), a polyethylene glycol (PEG), a polyhydroxyalkanoate (PHA), polyesters, poly(ortho esters), poly(anhydrides), poly(tyrosine carbonates), poly(imino carbonates), poly(phosphoesters), polyphosphazenes, poly(amino acids), polysaccharides, polyethers, polyamides, polyurethanes, polyalkylenes, polyalkylene oxides, a polyhydroxyalkanoate-polyethylene glycol copolymer. alkylene vinyl acetate copolymer, alkylene vinyl alcohol copolymers, or a mixture or blend of any two or more thereof.

In one embodiment, the hydrophilic polymer is poly(D,L-lactide), poly(L-lactide), polyglycolide, poly(D,L-lactide-co-glycolide), polycaprolactone, poly(D,L-lactide-co-caprolactone), poly(glycolide-co-caprolactone), poly(dioxanone), collagen, chitosan, alginate, polyethylene oxide, polypropylene oxide, polyethylene glycol (PEG), polyvinylpyrrolidone (PVP), poly(n-butyl methacrylate) (PBMA), poly(vinylidene fluoride-co-hexafluoropropene), or a mixture or blend of any two or more thereof.

In one embodiment, the hydrophilic polymer is hydroxymethylcellulose, hydroxyethylcellulose, hydroxypropylcellulose, hydroxypropylmethylcellulose or a mixture of any two or more thereof. In another embodiment, the hydrophilic polymer is hydroxypropylcellulose.

In any of the above embodiments, the distal end and the proximal end of the cylindrical body are sealed, the lumen forming a reservoir inside the cylindrical body. Alternatively, the distal end of the cylindrical body may be sealed and the proximal end of the cylindrical body is open. Alternatively, the distal end and the proximal end of the cylindrical body are open. Such cylindrical bodies may be formed by extrusion. Such cylindrical bodies may alternatively be formed by molding.

In any of the above embodiments, the ocular insert may have an outer surface of from about 0.1 mm² to about 200 mm². For example, this may include from about 0.5 mm² to about 200 mm², from about 1 mm² to about 200 mm², from about 2.5 mm² to about 200 mm², from about 5 mm² to about 200 mm², from about 10 mm² to about 100 mm², or from about 10 mm² to about 50 mm², or any subranges of any of these values.

In any of the above embodiments, the ocular insert may have a length of from about 1 mm to about 5 mm. This may be from about 1 mm to about 4 mm, from about 1 mm to about 3 mm, from about 1.5 mm to about 5 mm, from about 1.5 mm to about 4, from about 1.5 mm to about 3 mm, from about 2 mm to about 5 mm, from about 2 mm to about 4 mm, or from about 2 mm to about 3 mm, or any subranges of any of these values.

In any of the embodiments, the ocular insert may have a width of from about 1 mm to about 3 mm. For example, this may include from about 1 mm to about 2 mm, from about 1 mm to about 1.5 mm, from about 1.2 mm to about 3 mm, from about 1.5 mm to about 2 mm, from about 1.2 mm to about 1.5 mm, from about 1.3 mm to about 3 mm, from about 1.3 mm to about 2 mm, or from about 1.3 mm to about 1.5 mm, or any subranges of any of these values.

In any of the above embodiments, the ocular insert may have an outer diameter of from about 0.25 mm to about 3 mm. For example, this may include from about 0.50 mm to about 2.5 mm, about 0.75 mm to about 2.00 mm, about 1.00 mm to about 1.75 mm, or about 1.25 mm to about 1.5 mm, or any subranges of any of these values. In any of the above embodiments, the ocular insert has an outer diameter of about 1.29 mm.

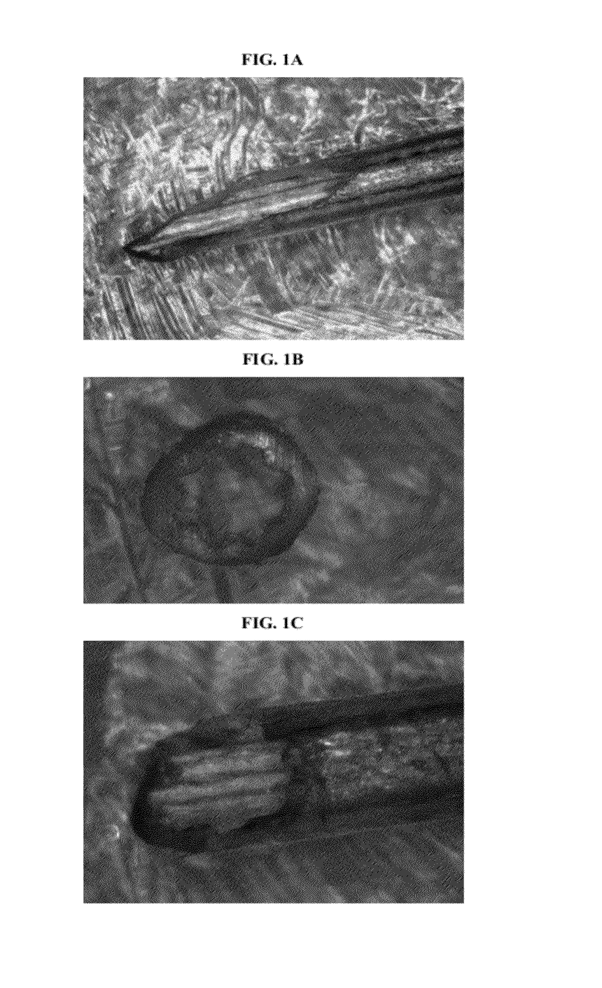

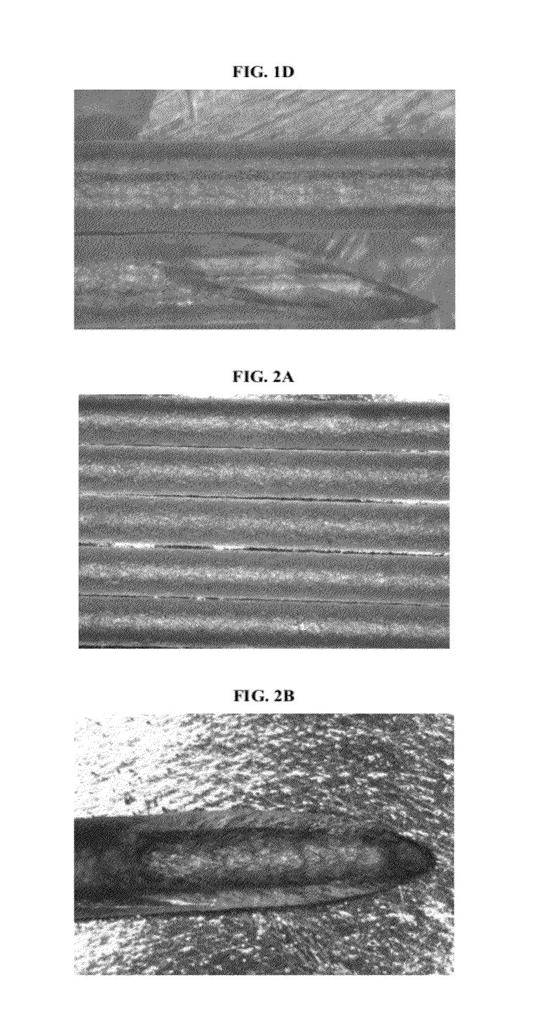

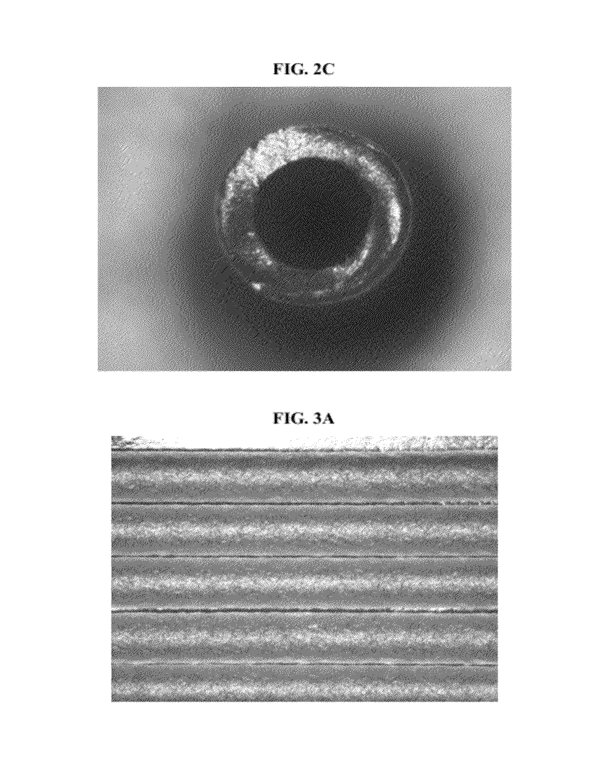

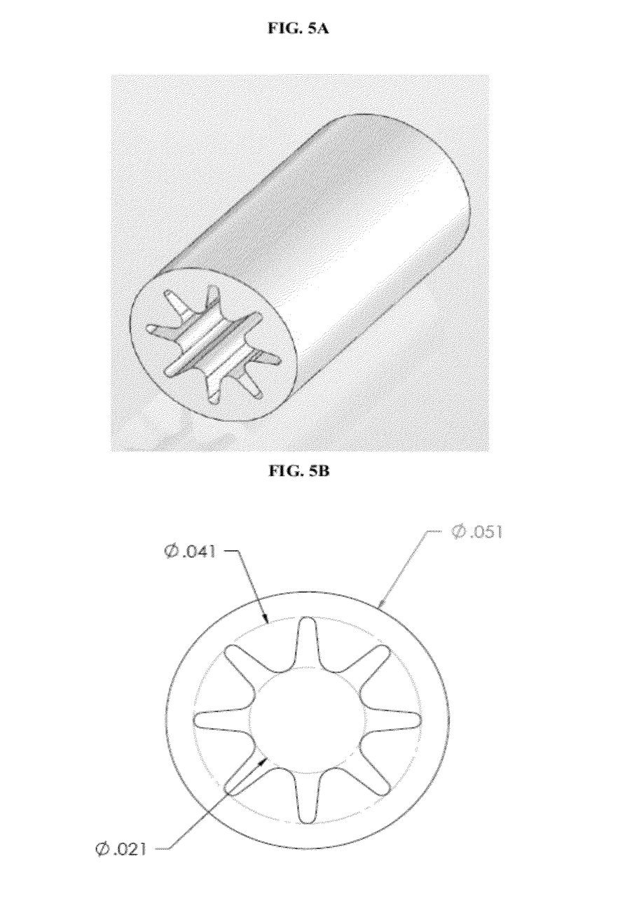

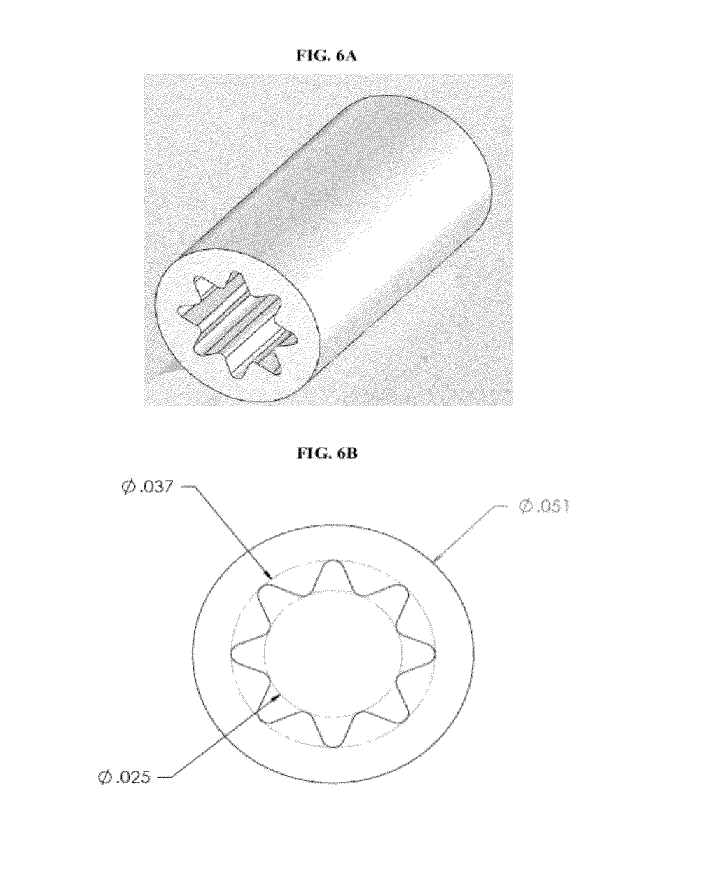

The ocular insert may include a single lumen or a plurality of lumens. The lumen or lumens may individually have a cross section which is round, circular, oval, star shaped, +shaped, or cog-shaped. In another embodiment, the at least one inner surface is grooved or ribbed, such as those illustrated in FIGS. 1A-1D, 5A-B, and 6A-B. “Grooved or ribbed tubes” include those ocular inserts which have a lumen having a star-shaped, “+”-shaped, or cog-shaped cross section. In another embodiment, the at least one inner surface is smooth. Such “smooth tube” ocular inserts are illustrated in FIGS. 2A-AC and 3A-3C.

The ocular insert may have an inner diameter of less than about 2.5 mm. This includes inner diameters which are less than about 2.25 mm, less than about 2 mm, less than about 1.75 mm, less than about 1.5 mm, less than about 1.25 mm, less than about 1 mm, or less than about 0.75 mm. In any of the above embodiments, the ocular insert has an inner diameter of less than about 1.25 mm. In another embodiment, the ocular insert has an inner diameter of about 0.75 mm.

The ocular insert may also include a pharmaceutically active agent, a dye, a lubricant, or an emollient, and, optionally a pharmaceutically acceptable excipients, such as a gelling agent. Illustrative gelling agents include, but are not limited to, gellan gum, polyethylene glycol (PEG), carboxymethyl cellulose (CMC) and the like and combinations thereof. The lumen may also include a pharmaceutically active agent, a dye, a lubricant, or an emollient, and, optionally a pharmaceutically acceptable excipient, such as a gelling agent.

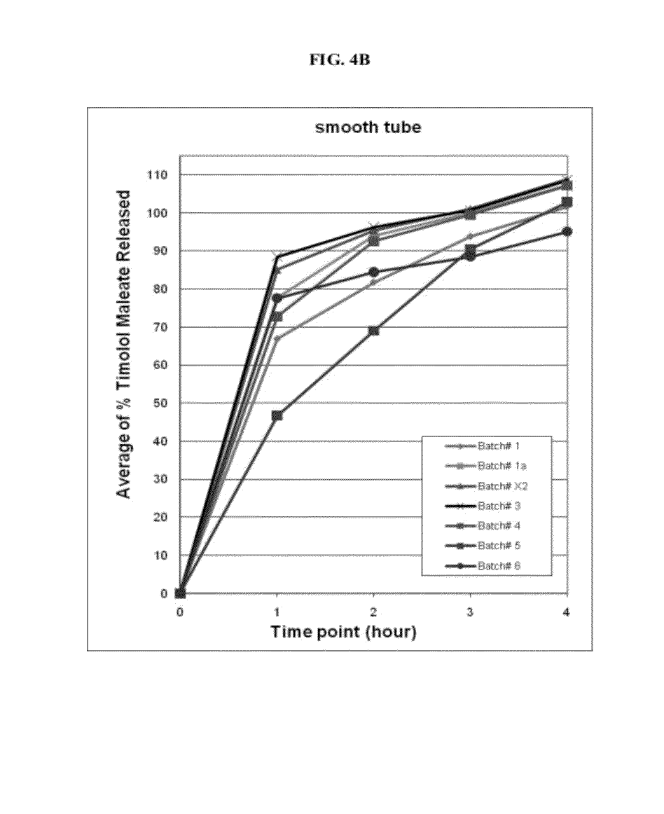

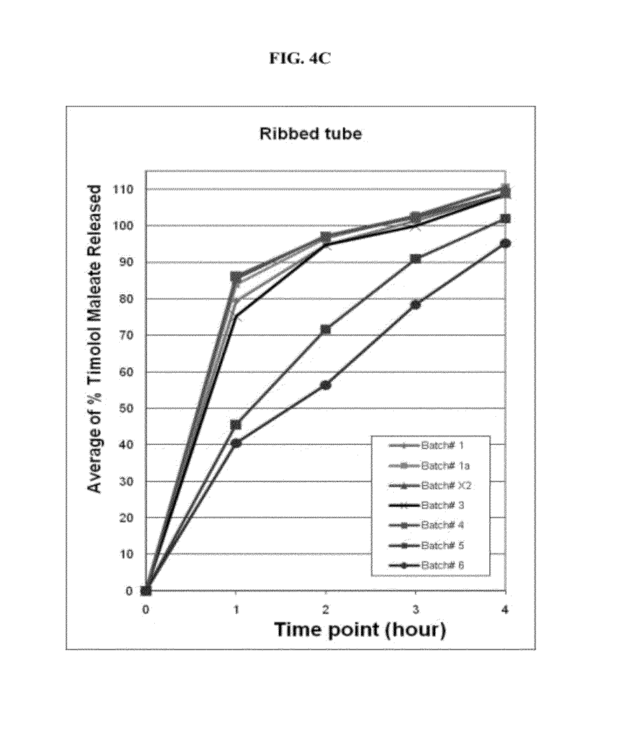

In another aspect, a composition is provided, the composition including an admixture of any of the above ocular inserts with a pharmaceutically active agent, a dye, a lubricant, an emollient, or a gelling agent. One illustrative example of such a composition are those utilized in the dissolution testing illustrated in FIGS. 4A-4C, which include an ocular insert with timolol maleate. The ocular inserts used in FIG. 4A include a tube loaded multiple times with an aqueous 0.5% ophthalmic solution of timolol maleate. The amount of timolol maleate in each of Batches 1-5 is shown in Table 1.

TABLE 1
Amount of Timolol Maleate in Multiply Filled Ocular Insert Batches

| | Formulation | | | | |
|---|---|---|---|---|---|
| | Timoptic 0.5% Ophthalmic Solution, | | | | |
| | Multiple Fill Injection MF) | | | | |
| | Batch # | | | | |
| | 1 | 2 | 3 | 4 | 5 |
| Theorhetical | 7.33 | 7.674 | 7.307 | 7.727 | 7.724 |
| Conc. | | | | | |
| (μg/mm) | | | | | |
| Acutal Conc. | 5.584 | 5.889 | 10.503 | 4.575 | 5.686 |
| (μg/mm) | | | | | |

The raw data from which FIG. 4A was generated is provided in the Table 2.

TABLE 2
Dissolution or Release Profile of Multiply Filled Timolol Maleate Inserts

| | % Released* (% RSD) | | | | | Assay (μg/mm) | | | | |
|---|---|---|---|---|---|---|---|---|---|---|
| | Time Point (hours) | | | | | Time Point (hours) | | | | |
| Formulation | 0 | 1 | 2 | 3 | 4 | 0 | 1 | 2 | 3 | 4 |
| 1 | 0 | 109.9 | 114.9 | 115.5 | 117.5 | 0 | 6.139 | 6.415 | 6.452 | 6.4562 |
| | | (11.0) | (6.9) | (7.0) | (7.0) | | | | | |
| 2 | 0 | 105.0 | 110.2 | 111.2 | 113.3 | 0 | 6.182 | 6.488 | 6.551 | 6.673 |
| | | (6.7) | (4.4) | (4.5) | (4.5) | | | | | |
| 3 | 0 | 82.1 | 82.8 | 83.0 | 84.6 | 0 | 8.625 | 8.699 | 8.716 | 8.888 |
| | | (7.6) | (8.4) | (9.0) | (9.0) | | | | | |
| 4 | 0 | 149.6 | 151.4 | 152.4 | 155.1 | 0 | 6.844 | 6.926 | 6.972 | 7.096 |
| | | (4.2) | (3.5) | (3.7) | (3.6) | | | | | |
| 5 | 0 | 139.1 | 139.9 | 141.5 | 143.8 | 0 | 6.366 | 6.399 | 6.472 | 6.578 |
| | | (4.0) | (4.2) | (4.2) | (4.1) | | | | | |

With the exception of Batch 3, substantially all the active agent loaded onto the ocular insert had been released in the first hour of the dissolution (or release) test.

The dissolution profiles, or release profiles, as graphically illustrated in FIGS. 4B and 4C, were generated from smooth tubes (4B) and ribbed tubes (4C) that were loaded with various formulations of timolol maleate containing various amounts of gelling agent, with the exception of Batch X2, which was loaded with an aqueous solution of timolol maleate (i.e., no gelling agent present). The components of each batch can be found in Table 3.

TABLE 3
Timolol Maleate Formulations Tested in Smooth and Ribbed Tubes

| | Formulations (% of total ingredients) | | | | | | |
|---|---|---|---|---|---|---|---|
| Ingredient | 1 | 1a | X2 | 3 | 4 | 5 | 6 |
| Timolol | 1.37 | 3.42 | 1.37 | 1.37 | 1.37 | 1.37 | 1.37 |
| Maleate | | | | | | | |
| Gellan Gum | 0.60 | 0.60 | — | — | — | — | — |
| *PEG 400 | — | — | — | 0.60 | 1.00 | — | — |
| **CMC | — | — | — | — | — | 0.60 | 1.00 |
| Water | 98.03 | 95.98 | 98.63 | 98.03 | 97.63 | 98.03 | 97.63 |
| Total | 100.00 | 100.00 | 100.00 | 100.00 | 100.00 | 100.00 | 100.00 |

*PEG is polyethylene glycol
**CMC is carboxymethylcellulose

The raw data from which FIG. 4B was generated is provided in Table 4.

TABLE 4
Average (n = 3) Release Data from Timolol-Loaded Smooth Tubes

| | % Released* (% RSD) | | | | | Assay (μg/mm) | | | | | |
|---|---|---|---|---|---|---|---|---|---|---|---|
| | Time Point (hours) | | | | | Time Point (hours) | | | | | **LOD |
| Form. | 0 | 1 | 2 | 3 | 4 | 0 | 1 | 2 | 3 | 4 | (%) |
| 1 | 0 | 66.9 | 81.7 | 93.8 | 101.6 | 0 | 5.022 | 6.140 | 7.041 | 7.627 | 3.30 |
| | | (6.5) | (3.8) | (1.9) | (1.1) | | | | | | |
| 1a | 0 | 77.7 | 94.0 | 100.1 | 107.3 | 0 | 14.561 | 17.613 | 18.762 | 20.103 | 3.23 |
| | | (2.8) | (1.8) | (1.2) | (0.8) | | | | | | |
| X2 | 0 | 85.1 | 95.4 | 101.1 | 108.8 | 0 | 6.617 | 7.415 | 7.862 | 8.463 | 3.13 |
| | | (4.5) | (0.3) | (0.5) | (0.9) | | | | | | |
| 3 | 0 | 88.5 | 96.2 | 100.7 | 108.6 | 0 | 6.996 | 7.606 | 7.956 | 8.577 | 3.17 |
| | | (4.8) | (1.9) | (2.1) | (2.1) | | | | | | |
| 4 | 0 | 72.8 | 92.5 | 99.5 | 107.1 | 0 | 5.625 | 7.154 | 7.690 | 8.283 | 3.17 |
| | | (5.2) | (3.0) | (2.2) | (1.4) | | | | | | |
| 5 | 0 | 46.7 | 69.0 | 90.5 | 102.9 | 0 | 3.850 | 5.690 | 7.453 | 8.479 | 4.70 |
| | | (5.2) | (1.7) | (0.8) | (1.0) | | | | | | |
| 6 | 0 | 77.6 | 84.4 | 88.5 | 95.1 | 0 | 4.755 | 5.179 | 5.427 | 5.831 | 3.87 |
| | | (21.5) | (20.9) | (20.8) | (21.2) | | | | | | |

*% Released was calculated based on actual assay value shown;
**Loss on drying, which shows the amount of water driven off at a specified temperature.

The raw data from which FIG. 4C was generated is provided in the Table 5.

TABLE 5
Average (n = 3) Release Data from Timolol-Loaded Ribbed Tubes

| | % Released* (% RSD) | | | | | Assay (μg/mm) | | | | | |
|---|---|---|---|---|---|---|---|---|---|---|---|
| | Time Point (hours) | | | | | Time Point (hours) | | | | | **LOD |
| Form. | 0 | 1 | 2 | 3 | 4 | 0 | 1 | 2 | 3 | 4 | (%) |
| 1 | 0 | 79.4 | 94.8 | 101.3 | 108.7 | 0 | 8.823 | 10.532 | 11.259 | 12.077 | 3.10 |
| | | (1.9) | (1.6) | (1.8) | (1.5) | | | | | | |
| 1a | 0 | 83.9 | 96.5 | 102.8 | 110.6 | 0 | 23.809 | 27.360 | 29.167 | 31.363 | 3.22 |
| | | (1.9) | (1.9) | (1.4) | (0.9) | | | | | | |
| X2 | 0 | 85.4 | 97.3 | 102.6 | 110.4 | 0 | 8.334 | 9.481 | 10.001 | 10.756 | 3.20 |
| | | (8.0) | (3.5) | (2.4) | (2.0) | | | | | | |
| 3 | 0 | 75.2 | 94.8 | 100.0 | 108.5 | 0 | 6.367 | 8.007 | 8.446 | 9.170 | 3.15 |
| | | (5.0) | (2.0) | (1.5) | (1.6) | | | | | | |
| 4 | 0 | 86.2 | 97.0 | 102.2 | 108.9 | 0 | 9.598 | 10.796 | 11.376 | 12.121 | 3.18 |
| | | (1.7) | (1.6) | (2.2) | (1.3) | | | | | | |
| 5 | 0 | 45.5 | 71.6 | 91.0 | 102.0 | 0 | 3.789 | 5.957 | 7.568 | 8.486 | 4.27 |
| | | (8.2) | (7.1) | (4.7) | (2.8) | | | | | | |
| 6 | 0 | 40.5 | 56.4 | 78.4 | 95.2 | 0 | 3.654 | 5.081 | 7.061 | 8.566 | 5.04 |
| | | (8.0) | (8.1) | (3.9) | (2.3) | | | | | | |

*% Released was calculated based on actual assay value shown;
**Loss on drying

In another aspect, a method of preparing the ocular insert is provided. The method includes extruding a strand of a biodegradable polymer along a longitudinal axis, the strand including at least one continuous lumen extending along the axis; cutting the strand into segments of a length suitable as an ocular insert, wherein the segments each including a distal end, and a proximal end.

In another aspect, a method of controlling the dissolution rate of an ocular insert having a cylindrical body with a lumen and a hydrophilic polymer is provided. The method includes varying a diameter of the lumen. It is contemplated that the dissolution rate of the ocular inserts can be modulated by varying the thickness of the ocular inserts provided here. The dissolution rate decreases, when the thickness of the ocular insert is increased. As used herein, “thickness” refers to a distance from the inner surface to the outer surface of the ocular insert, which is dependent upon the diameter of the lumen or the inner diameter of the ocular insert. Conversely, when the thickness is decreased, the dissolution rate is increased. The hydrophilic polymer included in the ocular insert may have a molecular weight from about 80,000 to about 1,150,000. In some embodiments, the molecular weight of the hydrophilic polymer is from about 200,000 to about 900,000. In other embodiments, the molecular weight of the hydrophilic polymer is from about 400,000 to about 700,000.

The dissolution rate of the ocular inserts may be modulated by varying the molecular weight of the hydrophilic polymer. For example, and without limitation, the dissolution rate can be decreased by employing a higher molecular weight polymer compared to a lower molecular weight polymer having similar monomers and polymeric attachment. The dissolution rate may also be varied by adjusting the viscosities of the fill media. For example, low cps CMC may provide a more desirable rate of dissolution or release than water alone.

In another aspect, a method of treating an eye disorder is provided, the method including depositing an ocular insert provided herein into the conjunctival cul-de-sac of an eye in need thereof, wherein the ocular insert has a dissolution rate that is increased in comparison to an ocular insert including the hydrophilic polymer with which does not include a lumen.

In another aspect, a method of manufacture of the ocular insert is provided, the method including contacting the ocular insert with a solvent or a solution to a provide a solvated ocular insert. The solution may include a biodegradable hydrophilic polymer. The solution may include a plasticizer.

The solvent or the solution may include an aqueous solvent or an organic solvent. A variety of solvents may be used. The solvent is not particularly limited provided the solvent is capable of dissolving the desired pharmaceutically active agent. The phrase “capable of dissolving” is meant to include those solvents that completely dissolve and those that partially dissolve the desired pharmaceutically active agent. An illustrative solvent is water. Illustrative organic solvents include, but are not limited to, methanol, ethanol, n-propanol, iso-propanol, n-butanol, iso-butanol, tert-butanol, dimethyl ether, methylethyl ether, methyl-tert-butyl ether, diethyl ether, ethylene glycol, propylene glycol, dioxane, dimethyl sulfoxide, dimethyl formamide or mixtures of any two or more such solvents. The only limitation regarding the solvent is that it be susceptible to being dried from the polymer by evaporation or vacuum. In some embodiments, the solvent is deionized water or a buffered system.

In another embodiment, the solution includes a pharmaceutically active agent, e.g., and without limitation as disclosed herein, a dye, a lubricant, or an emollient dissolved in a pharmaceutically acceptable liquid carrier.

In one embodiment, the method may also include allowing the solvated ocular insert to collapse to provide a strip including a biodegradable polymer and, optionally, a payload of a pharmaceutically active agent. The strip may be a flattened strip or a twisted strip.

In another embodiment, the strips as prepared are provided herein. Such strips may have lengths and widths the same as or similar to those of the cylindrical ocular inserts disclosed herein.

In another aspect, a method of treating an eye disorder is provided. The method of treatment includes contacting the strip provided herein with an eye of a patient having an eye disorder. It is not required to remove a residual ocular insert or a residual strip from the patient's eye. As used herein, a “residual ocular insert” or “a residual strip” refers to an ocular insert or a strip used for treating an eye disorder, which has eroded partly and/or have completely or partly lost the pharmaceutically active agent.

In another aspect, a method of releasing a pharmaceutically active agent into a liquid medium, the method including contacting the ocular insert containing a pharmaceutically active agent provided herein, or the composition provided herein, or the strip containing a pharmaceutically active agent provided herein, with a liquid medium, thereby releasing the pharmaceutically active agent to the liquid medium. In some embodiments, the ocular inserts and strips are capable of releasing the active pharmaceutical ingredient over extended periods of time from 1 hour up to 24 hours. In another embodiment, the liquid medium is a body fluid. In another embodiment, the body fluid includes tears. In another embodiment, the contacting is performed in vitro or in vivo.

In some embodiments, the identification of the pharmaceutically active agent released from the ocular drug delivery device is performed in vitro or in vivo. When performed in vitro such methods may be used for identifying active pharmaceuticals agents that can be, or need to be, delivered to the eye at a certain rate. When performed in vivo, such methods may be used as animal models of ocular drug delivery. When released in vivo in humans, the method may lead to an alleviation of one or more negative effects associated with a medical condition, especially an ophthalmic condition when released in or on a human eye. The in vitro dissolution profiles of a pharmaceutically active agent, from cylindrical ocular inserts loaded with the pharmaceutically active agent, are determined following procedures well known to the skilled artisan. For example, and not for limitation, dissolution profiles for ocular inserts loaded with timolol maleate are provided herewith in the attached FIGs.

As noted above, the ocular inserts and/or the strips include a therapeutically effective amount of the pharmaceutically active agent. By “therapeutically effective amount” it is meant that amount which results in a desired therapeutic effect for a particular eye disorder. As further described below, a desired therapeutic effect may be the alleviation or amelioration, in whole or in part, of symptoms associated with the eye disorder; the halting of further progression or worsening of those symptoms; or the prevention of the eye disorder. As described above, the amount needed to achieve these effects may be readily determined by considering the relevant factors (e.g., nature of the eye disorder, the subject's characteristics, dosing regimen, etc.).

The pharmaceutically active agent is active in treating disorders of the eye. Illustrative the pharmaceutically active agents include, but are not limited to, a macrolide antibiotic, a fluoroquinolone antibiotic, an antibacterial, an antiviral, a prostaglandin, an antiallergenic, a non-steroidal anti-inflammatory, a steroidal anti-inflammatory, a decongestant, a miotic, an anti-cholinesterase, a mydriatic, a sympathomimetic, a prostaglandin inhibitor, a rho kinase inhibitor, a β-blocker, cyclosporine, a P2Y2 agonist, resolvins such as docosahexanoic acid or eicosapentanoic acid, or a mixture of any two or more thereof. Non-limiting examples of the pharmaceutically active agents include Acebutolol, Acyclovir, Betaxolol, Bimatoprost, Bilastine, Brimonidine Tartrate, Brinzolamide, Bromfenac Sodium, Cefazolin, Cephalexin, Cephadroxil, Ciprofloxacin, Ciprofloxacin HCl, Cyclosporine, Dexamethasone, Dorzolamide HCl, Epinastine HCl, Erythromycin, Gancicylovir, Gatifloxacin, Gentamicin Sulfate, Ketorolac Tromethamine, Labetalol, Latanoprost, Loteprednol Etabonate, Moxifloxacin HCl, Nepafenac, Ofloxacin, Olopatadine HCl, Penicillin, Pindolol, Prednisolone, Propanolol, polymyxin B Sulfate/Trimethoprim Sulfate, Sulfacetamide Sodium, Timolol Maleate, Triflourodine, Tobramycin, Travoprost, Vancomycin, or a mixture of any two or more thereof.

Other non-limiting examples of the pharmaceutically active agents include, Azelastine HCl, Atropine sulfate, Betamethasone, Carbachol, Pheniramine, Cromolyn sodium, Cyclopentolate, Demecarium bromide, Dexamethasone 21-phosphate, Erythromycin Base, Fluorometholone, Gatifloxacin, Homatropine, Hydroxyamphetamine, Idoxuridine, Medrysone, Methylprednisolone, Naphazoline, Resolvins, Phospholipids, Phenylephrine, Phospholine iodide, Prednisolone Acetate, Prednisolone Sodium Sulfate, Sulfisoxazole, Tetrahydrazoline HCl, Timolol, Tobramycin Sulfate, Tropicamide, 6-hydroxy-2-sulfamoylbenzo[b]thiophene, 6-acetoxy-2-sulfamoylbenzo[b]thiophene, 5,6-dihydro-4H-4-hydroxythieno[2,3-b]thiopyran-2-sulfonamide-7,7-dioxide, or a mixture of any two or more thereof or a mixture of any one or more thereof with one or more pharmaceutically active agents of the previous paragraph.

In another embodiment, pharmaceutically active agent is Acyclovir, Antazoline, Aspirin, Atropine sulfate, Betamethasone, Bimatoprost, Brimonidine Tartrate, Brinzolamide, Bromfenac Sodium, Carbachol, Chlorpheniramine, Ciprofloxacin HCl, Cyclopentolate, Cyclosporine, Demecarium bromide, Dexamethasone, Dexamethasone 21-phosphate, Di-isopropyl fluorophosphate, Dorzolamide HCl, Epinastine HCl, Epinephrine, Erythromycin Base, Eserine, Eucatropine, Fluocinolone, Fluorometholone, Gatifloxacin, Gentamicin Sulfate, Homatropine, Hydroxyamphetamine, Hydrocortisone, Hydrocortisone acetate, Idoxuridine, Indomethacin, Ketorolac Tromethamine, Latanoprost, Loteprednol Etabonate, Medyrisone, Methapyriline, Methylprednisolone, Moxifloxacin HCl, Naphazoline, Nepafenac, Nitrofurazone, Ofloxacin, Olopatadine HCl, Phospholine iodide, polymyxin B Sulfate, Prednisolone Acetate, Prednisolone Sodium Sulfate, Prophenpyridamine, Propanolol, Pyrilamine, Scopolamine, Sulfacetamide Sodium, Sulfamethizole, Sulfisoxazole, Sulindac, Tetrahydrazoline, Timolol, Timolol Maleate, Tobramycin, Tobramycin Sulfate, Travoprost, Travoprost (Benzalkonium), Triamcinolone, Tropicamide, Vancomycin, 6-hydroxy-2-sulfamoylbenzo[b]thiophene, 6-acetoxy-2-sulfamoylbenzo[b]thiophene, 5,6-dihydro-4H-4-hydroxythieno[2,3 -b]thiopyran-2-sulfonamide-7,7-dioxide, or a mixture of any two or more agents.

The present ocular inserts and strips may be readily be prepared at lower temperatures or under much more gentle conditions than are present in traditional extrusion processes. Accordingly, the present methods and compositions are amenable to situations where the pharmaceutically active agent may be heat sensitive, poorly soluble, soluble, or very soluble in an aqueous medium, or is in the form of a micronized particle, a nano-particle, an emulsion or a colloidal solution.

Illustrative eye disorders that may be treated with the ocular inserts or strips include, but are nto limited to, dry eye, infections caused by bacteria, viruses, or surgical procedures, glaucoma, ocular melanoma, retinitis pigmentosa, elevated intraocular pressure, photoreceptor degeneration, intraocular neovascularization, vitreoretinopathy, retinal degeneration, retinal ischemia, retinal neovascularization, retinal pigment epithelium disease, dry eye syndrome, seasonal allergies, trachoma, a dry eye syndrome including meibumium gland dysfunction or aqueous deficient dry eye, viral keratitis and bacterial keratitis.

As another non-limiting example, the cellulose polymer of the disclosed ocular inserts and strips may include a substantial amount of hydroxypropyl cellulose. Such devices are distinguished from conventional devices which seek to limit the amount of hydroxypropyl cellulose because of the potential for this polymer to cause blurred vision. In some embodiments, the polymer includes more than 30% hydroxypropyl cellulose. In other embodiments, the polymer consists essentially of hydroxypropyl cellulose.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

The use of the terms “a” and “an” and “the” and similar referents in the context of describing the elements (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential to the practice of the invention.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Additionally the phrase “consisting essentially of” will be understood to include those elements specifically recited and those additional elements that do not materially affect the basic and novel characteristics of the claimed invention. The phrase “consisting of” excludes any element not specified. In addition, where features or aspects of the disclosure are described in terms of Markush groups, those skilled in the art will recognize that the disclosure is also thereby described in terms of any individual member or subgroup of members of the Markush group.

The present invention, thus generally described, will be understood more readily by reference to the following examples, which are provided by way of illustration and are not intended to be limiting of the present invention.

### EXAMPLES

### Example 1

Preparation of a cylindrical ocular insert having a round lumen. This example demonstrates the preparation of cylindrical ocular inserts (or tubes) having a round lumen by extruding hydroxypropyl cellulose (HPC) and cutting the extruded tubes.

### Materials Used

Hydroxy propyl Cellulose.

### Equipment Used

Hot melt extruder equipped with a mandrel, and short land die and long land die.

### Experimental Description

The HPC was dried at a temperature of 65° C. in a convection oven or fluid bed dryer. Loss on drying was analyzed before and after drying and as well as before the actual extrusion process, are presented in Table 6.

TABLE 6

| Tray Identity | Before Drying | After Drying | Before Extrusion |
|---|---|---|---|
| Bottom Tray | 2.2% | 0.019% | N/A |
| Middle Tray | | 0.041% | N/A |
| Top Tray | | 0.018% | 0.114% |

HPC was extruded maintaining a die head to outer diameter (OD) ratio of 1:1. Extruded tubes were cut to approximately 91.4 cm and collected online and then were cut manually to a 3.5 mm length. The average OD and inner diameter (ID) were measured. Representative dimensions for Sample No. 1, prepared using a Tool 1 (short land die) are tabulated below. FIGS. 2A-2C illustrate the outer surface, the skived end, and the cross section of Sample No. 1, an ocular insert, whose ID and OD are shown in Table 7.

TABLE 7

| | | | Wall |
|---|---|---|---|
| Sample | ID | OD | Thickness |
| No. | (mm) | (mm) | (mm) |
| 1 | 0.787 | 1.285 | 0.249 |
| 2 | 0.775 | 1.278 | 0.251 |
| 3 | 0.787 | 1.275 | 0.251 |
| 4 | 0.787 | 1.285 | 0.249 |
| 5 | 0.775 | 1.270 | 0.249 |
| Ave | 0.782 | 1.278 | 0.249 |

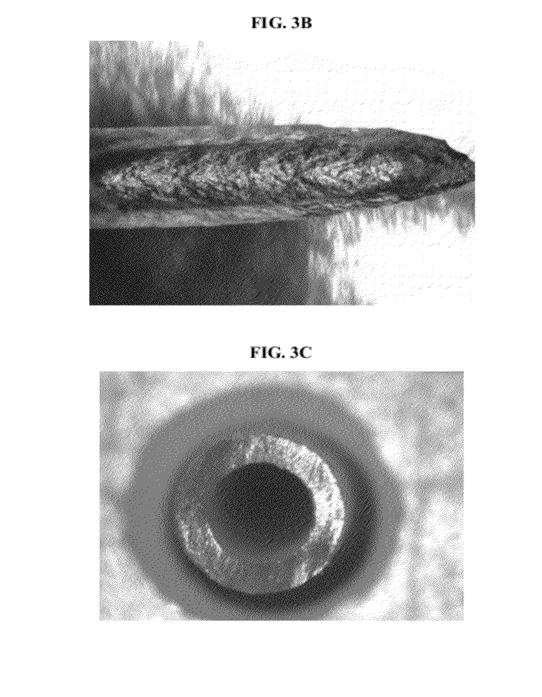

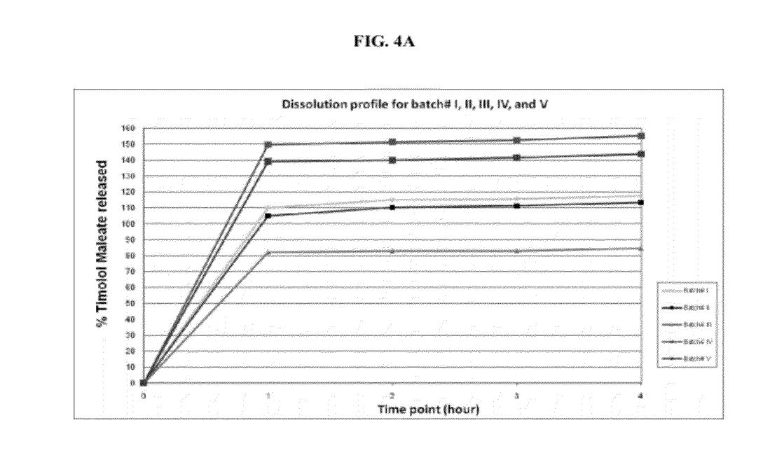

Representative dimensions for Sample No. 5, prepared using a Tool 2 (long land die) are found in Table 8. FIGS. 3A-3C illustrate the outer surface, the skived end, and the cross section of sample No. 5, an ocular insert.

TABLE 8

| | | | Wall |
|---|---|---|---|
| Sample | ID | OD | Thickness |
| No. | (mm) | (mm) | (mm) |
| 6 | 0.762 | 1.267 | 0.254 |
| 7 | 0.775 | 1.270 | 0.249 |
| 8 | 0.775 | 1.285 | 0.257 |
| 9 | 0.787 | 1.285 | 0.249 |
| 10 | 0.787 | 1.288 | 0.251 |
| Ave | 0.777 | 1.280 | 0.251 |

Cylindrical ocular inserts, having a round, circular lumen, and an inner and outer surface were prepared in accordance to the methods described above. A change in die head temperature influenced the physical property of the prepared ocular inserts. The material was more brittle at colder temperature and was more flexible at higher temperature.

### Example 2

Preparation of a cylindrical ocular insert having a cog shaped lumen and a smooth inner surface. This example demonstrates the preparation of cylindrical ocular inserts (or tubes) having a cog shaped lumen with 8 teeth and a smooth inner surface by extruding hydroxypropyl cellulose (HPC) and cutting the extruded tubes.

Hydroxypropylcellulose (HPC) was dried at a temperature of 65° C. in the convention tray dryer, and then packed in moisture-proof foil lined bags by purging high purity nitrogen gas, and heat sealed. A (¾″) single screw extruder (hot melt extruder equipped with a cog shaped mandrel with 8 teeth) was used to extrude the HPC material. The hopper was manually filled and nitrogen purge was connected to the extruder hopper. The extrusion process was started, and once the desired temperatures were reached, the extruder was allowed to stabilize and then samples were collected. The OD and ID of a number of manufactured cylindrical ocular inserts having a cog shaped lumen and a smooth inner surface in Table 9.

TABLE 9

| | | | Wall |
|---|---|---|---|
| Sample | | | Thickness |
| No. | OD (mm) | ID (mm) | (mm) |
| 11 | 1.273 | 0.762 | 0.254 |
| 12 | 1.283 | 0.762 | 0.262 |
| 13 | 1.285 | 0.762 | 0.262 |
| 14 | 1.285 | 0.762 | 0.262 |
| 15 | 1.285 | 0.762 | 0.262 |
| 16 | 1.285 | 0.762 | 0.262 |
| 17 | 1.285 | 0.762 | 0.262 |
| 18 | 1.285 | 0.762 | 0.262 |
| 19 | 1.283 | 0.762 | 0.259 |
| 20 | 1.283 | 0.762 | 0.262 |
| Avg | 1.283 | 0.762 | 0.262 |

TABLE 10

| | | | Wall |
|---|---|---|---|
| Sample | | | Thickness |
| No. | OD (mm) | ID (mm) | (mm) |
| 21 | 1.293 | 0.686 | 0.305 |
| 22 | 1.290 | 0.711 | 0.290 |
| 23 | 1.288 | 0.711 | 0.295 |
| 24 | 1.290 | 0.711 | 0.297 |
| 25 | 1.293 | 0.711 | 0.297 |
| 26 | 1.283 | 0.711 | 0.292 |
| 27 | 1.283 | 0.711 | 0.292 |
| 28 | 1.288 | 0.711 | 0.295 |
| 29 | 1.285 | 0.711 | 0.292 |
| 30 | 1.283 | 0.711 | 0.292 |
| Avg | 1.288 | 0.708 | 0.295 |

TABLE 11

| | | | Wall |
|---|---|---|---|
| Sample | | | Thickness |
| No. | OD (mm) | ID (mm) | (mm) |
| 31 | 1.270 | 0.787 | 0.246 |
| 32 | 1.270 | 0.787 | 0.249 |
| 33 | 1.265 | 0.787 | 0.243 |
| 34 | 1.265 | 0.787 | 0.246 |
| 35 | 1.270 | 0.787 | 0.249 |
| 36 | 1.262 | 0.787 | 0.243 |
| 37 | 1.267 | 0.787 | 0.246 |
| 38 | 1.270 | 0.787 | 0.249 |
| 39 | 1.267 | 0.787 | 0.246 |
| 40 | 1.267 | 0.787 | 0.246 |
| Avg | 1.267 | 0.787 | 0.246 |

TABLE 12

| | | | Wall |
|---|---|---|---|
| Sample | | | Thickness |
| No. | OD (mm) | ID (mm) | (mm) |
| 41 | 1.273 | 0.787 | 0.243 |
| 42 | 1.262 | 0.787 | 0.239 |
| 43 | 1.270 | 0.787 | 0.241 |
| 44 | 1.278 | 0.787 | 0.243 |
| 45 | 1.270 | 0.787 | 0.241 |
| 46 | 1.273 | 0.787 | 0.241 |
| 47 | 1.270 | 0.787 | 0.241 |
| 48 | 1.270 | 0.787 | 0.241 |
| 49 | 1.278 | 0.787 | 0.246 |
| 50 | 1.273 | 0.787 | 0.243 |
| Avg | 1.272 | 0.787 | 0.242 |

TABLE 13

| | | | Wall |
|---|---|---|---|
| Sample | | | Thickness |
| No. | OD (mm) | ID (mm) | (mm) |
| 51 | 1.275 | 0.864 | 0.213 |
| 52 | 1.270 | 0.864 | 0.203 |
| 53 | 1.252 | 0.864 | 0.201 |
| 54 | 1.262 | 0.864 | 0.206 |
| 55 | 1.270 | 0.864 | 0.211 |
| 56 | 1.267 | 0.864 | 0.208 |
| 57 | 1.267 | 0.864 | 0.208 |
| 58 | 1.273 | 0.864 | 0.211 |
| 59 | 1.280 | 0.864 | 0.216 |
| 60 | 1.280 | 0.864 | 0.213 |
| Avg | 1.270 | 0.864 | 0.209 |

A cog shaped die tool was successfully employed to prepare cylindrical ocular inserts having a cog shaped lumen and a smooth inner surface. The ID and wall thickness was varied by changing internal air pressure and puller pressure.

### Example 3

Preparation of drug loaded tubes. Timolol maleate ophthalmic solution and timolol maleate gel forming solution were injected into the tubes manufactured from examples 1 and 2. In certain examples, 91.5 cm (about 36 inches) long tubes were injected with drug solution, gel forming solution, or formulations containing gelling agents, as described elsewhere herein. The drug loaded ocular inserts were dried at room temperature between 2 to 24 hours. It is important to note that tubes longer that about 30.5 cm (about 12 inches) loaded with liquid solvent, solution, composition, or formulation would more likely than not collapse into strips. Tubes up to about 30.5 cm long would generally not collapse after loading, however. Smooth tubes would collapse into twisted strips, while ribbed tubes would collapse into substantially flat strips.

### Example 4

Preparation of ocular strips. A cylindrical ocular insert, prepared as discussed above, is filled with an aqueous or an organic solvent, suspension, micro-emulsion or nano-emulsion optionally in presence of a pharmacologically active agent. The filled tubes are dried at room temperature or in an oven between 2 to 24 hours. The tubes having the requisite length would collapse to form a thin strip and the aqueous or organic solvent permeates through the tube wall and evaporates. The tube flattens into a strip in about 20 minutes to about 24 hrs with or without twisting. The strips can then be cut to a desirable size, typically, 3.0-4.0 mm, preferably 3.5 mm in length.

### Example 5

In Vivo Study of pharmaceutically active agent-loaded ocular insert tube or strip in animal models of eye disease. An ocular insert tube or strip containing hydroxypropyl cellulose will be used as the ocular delivery system. The ocular insert tube or strip will be loaded with a pharmaceutically active agent directed to the treatment of an eye disease. Animals modeling human diseases will be selected for uniformity of condition based on an appropriate grading standard and divided into control and experimental groups. Unloaded and drug-loaded, ocular inserts (tubes and strips) will be administered to control and experimental animals, respectively. Following an appropriate period of treatment, animals will be subjectively evaluated for the extent of disease regression. The animals will then be euthanized and the relevant tissues examined histologically. Relevant parameters will be tabulated and compared to assess the efficacy of the drug.

Animals used to model human eye diseases include mice, rats, dogs, cats, rabbits, monkeys, pigs, and guinea pigs. Potential diseases that may be modeled include, but are not limited to inflammatory eye diseases, neoplastic disorders, retinitis pigmentosa, elevated intraocular pressure, photoreceptor degeneration, intraocular neovascularization, vitreoretinopathy, retinal degeneration, retinal ischemia, retinal neovascularization, retinal pigment epithelium disease, and trachoma.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An ocular insert having a cylindrical body comprising:
a proximal end;
a distal end;
a longitudinal axis defining a length of the cylindrical body from the proximal end to the distal end;
an outer surface;
an inner surface defining a lumen extending along the longitudinal axis;
a transverse axis, orthogonal to the longitudinal axis and defining a width of the cylindrical body; and
a hydrophilic polymer.

2. The ocular insert of claim 1 in which the hydrophilic polymer comprises hydroxypropyl cellulose, hydroxypropylmethylcellulose, hydroxymethylcellulose, 2-hydroxyethyl starch, poly(Bisphenol A-iminocarbonate), poly(butylenes succinate), poly(butylenes succinate-co-adipate), poly(butylene adipate-co-terephthalate), poly[1,6-bis(p-carboxyphenoxy)hexane], poly[(1,6-bis(p-carboxyphenoxy)hexane)-co-sebacic acid], poly(bis(4-carboxyphenoxy)phosphazene), poly(bis(1,4-dioxapentyl)phosphazene), poly(bis(1-(ethoxycarbonyl)methylamino)phosphazene), poly[bis(1-(ethoxycarbonyl)-2-phenylethylamino)phosphazene], poly[1,4-bis(hydroxyethyl)terephthalate-alt-ethyloxyphosphate], poly[1,4-bis(hydroxyethyl)terephthalate-alt-ethyloxyphosphate]-co-1,4-bis(hydroxyethyl)terephthalate-co-terephthalate, poly(caprolactone) (PCL), polycaprolactone diol, polycaprolactone triol, polycaprolactone-polyglycolide, poly(ε-caprolactone)-block-poly(ethylene glycol)-block-poly(ε-caprolactone) (PCL-b-PEG-b-PCL), polycaprolactone-block-polytetrahydrofuran-block-polycaprolactone, poly(detosu-1.6 HD-t-CDM ortho ester), poly(p-dioxanone) (PDS), polydioxanone-polyglycolide-poly(trimethylene carbonate), Biosyn, poly(DTH imino carbonate), poly(epsilon-caprolactone) (PCL), polyesteramide (PEA), poly(ethylene glycol)-block-poly(ε-caprolactone) methyl ether (PCL-b-PEG), poly(ethylene oxide)-block-polycaprolactone (4-arm), poly(ethylene oxide)-block-polylactide (4-arm), polylactide-block-poly(ethylene glycol)-block-polylactide poly(-ethyl-1-glutamate), polyglycolide (PGA), poly(glycolide-co-caprolactone) Monocryl, poly(glycolide-co-L(−)lactide) Vicryl, polyglycolide-poly(trimethylene carbonate) Maxon, polyglycolide-polycaprolactone-poly(trimethylene carbonate)-polyglycolide Caprosyn, polyhydroxybutyrate (PHB), poly[(R)-3-hydroxybutyric acid], poly(3-hydroxybutyrate-co-3-hydroxyhexanoate) PHBHx, poly(hydroxybutyrate-co-hydroxyoctanoate) PHBOd, poly(3-hydroxybutyrate-co-3-hydroxyoctadecanoate) PHBO, poly(3-hydroxybutyrate-co-3-hydroxyvalerate) PHBV, Biopol, poly[L(−)lactide] (PLA), polylactic acid-polyethylene glycol (PLEG), poly(DL-lactide-co-caprolactone), poly(lactide-co-glycolide) PLGA, poly(L-lactide-co-caprolactone-co-glycolide), (PLGC), poly[(lactide-co-ethylene glycol)-co-ethyloxyphosphate], poly(sebacic acid) (SA), poly(sebacic acid-hexadecandioic acid anhydride), poly(trimethylenecarbonate) (PTMC), poly(hydroxy ester ether), polyhydroxyalkanoates (PHA), poly(L-lactide-co-dl-lactide), L-PLA-DL-PLA (Inion), poly(L-lactide-co-DL-lactide-co-Trimethylenecarbonate), L-PLA-D-PLA-TMC (Inion) or combinations thereof.

3. The ocular insert of claim 1 in which the hydrophilic polymer is hydroxypropylcellulose.

4. The ocular insert of claim 1 in which the distal end and the proximal end of the cylindrical body are sealed, the lumen forms a reservoir in the interior of the cylindrical body.

5. The ocular insert of claim 1 in which the distal end of the cylindrical body is sealed and the proximal end of the cylindrical body is open.

6. The ocular insert of claim 1 in which the distal end and the proximal end of the cylindrical body are open.

7. The ocular insert of claim 1 in which the outer surface has a surface area ranging from about 0.1 mm² to about 200 mm².

8. The ocular insert of claim 1 comprising one lumen.

9. The ocular insert of claim 1 comprising a plurality of lumens.

10. The ocular insert of claim 1 in which the at least one lumen has a cross section, which is circular, oval, star shaped, +shaped, or cog shaped with 3-8 cogs.

11. The ocular insert of claim 1 further comprising a pharmaceutically active agent, and, optionally a pharmaceutically acceptable excipient.

12. The ocular insert of claim 11 in which the pharmaceutically active agent is an antibiotic, an anti-bacterial, an anti-viral, an anti-allergenic, a non-steroidal anti-inflammatory, a steroidal anti-inflammatory, a decongestant, a miotic, an anti-cholinesterase, a mydriatic, a sympathomimetic, a β-blocker, or a mixture of any two or more thereof.

13. The ocular insert of claim 11 in which the pharmaceutically active agent is Acyclovir, Antazoline, Aspirin, Atropine sulfate, Betamethasone, Bimatoprost, Brimonidine Tartrate, Brinzolamide, Bromfenac Sodium, Carbachol, Chlorpheniramine, Ciprofloxacin HCl, Cyclopentolate, Cyclosporine, Demecarium bromide, Dexamethasone, Dexamethasone 21-phosphate, Di-isopropyl fluorophosphate, Dorzolamide HCl, Epinastine HCl, Epinephrine, Erythromycin Base, Eserine, Eucatropine, Fluocinolone, Fluorometholone, Gatifloxacin, Gentamicin Sulfate, Homatropine, Hydroxyamphetamine, Hydrocortisone, Hydrocortisone acetate, Idoxuridine, Indomethacin, Ketorolac Tromethamine, Latanoprost, Loteprednol Etabonate, Medyrisone, Methapyriline, Methylprednisolone, Moxifloxacin HCl, Naphazoline, Nepafenac, Nitrofurazone, Ofloxacin, Olopatadine HCl, Phospholine iodide, polymyxin B Sulfate, Prednisolone Acetate, Prednisolone Sodium Sulfate, Prophenpyridamine, Propanolol, Pyrilamine, Scopolamine, Sulfacetamide Sodium, Sulfamethizole, Sulfisoxazole, Sulindac, Tetrahydrazoline, Timolol, Timolol Maleate, Tobramycin, Tobramycin Sulfate, Travoprost, Travoprost (Benzalkonium), Triamcinolone, Tropicamide, Vancomycin, 6-hydroxy-2-sulfamoylbenzo[b]thiophene, 6-acetoxy-2-sulfamoylbenzo [b]thiophene, 5,6-dihydro-4H-4-hydroxythieno [2,3-b]thiopyran-2-sulfonamide-7,7-dioxide, or a mixture of any two or more thereof.

14. The ocular insert of claim 1 in which the lumen includes a pharmaceutically active agent, and, optionally a pharmaceutically acceptable excipient.

15. The ocular insert in claim 14 in which the pharmaceutically active agent is heat sensitive, poorly soluble, soluble, very soluble, a micronized particle, a nano-particle, an emulsion or a colloidal solution.

16. The ocular insert of claim 14 in which the pharmaceutically active agent is an antibiotic, an anti-bacterial, an anti-viral, an anti-allergenic, a non-steroidal anti-inflammatory, a steroidal anti-inflammatory, a decongestant, a miotic, an anti-cholinesterase, a mydriatic, a sympathomimetic, a β-blocker, or a mixture of any two or more thereof.

17. A method of preparing an ocular insert comprising extruding a strand of a biodegradable polymer along a longitudinal axis, the strand comprising at least one continuous lumen extending along the longitudinal axis; cutting the strand into segments of a length suitable as an ocular insert, wherein the segments each comprise a distal end, and a proximal end.

18. The method of claim 17 in which the cylindrical body contains therein a pharmaceutically active agent, and optionally a pharmaceutically acceptable excipient.

19. A method of treating an eye disorder, comprising depositing an ocular insert into the conjunctival cul-de-sac of an eye in need thereof, the ocular insert having a cylindrical body comprising a lumen and a hydrophilic polymer, wherein the ocular insert has a dissolution rate that is increased in comparison to an ocular insert comprising the hydrophilic polymer but not including the lumen.
